SEQUENCE LISTING (3 sequences)

SEQ ID NO: 1 - protein, 30 aa, Listeria sp.
VNAENPPAYISSVAYGRQVYLKSTNSHSTK

SEQ ID NO: 2 - protein, 31 aa, Listeria sp.
VNAENPPAYISSSAYGRQVYLKLSTNSHSTK

SEQ ID NO: 3 - protein, 9 aa, Listeria sp.
SAYGRQVYL

(19) United States
(12) Patent Application Publication
(10) Pub. No.: US 2026/0081024 A1
(43) Pub. Date: Mar. 19, 2026

(54) SYSTEMS AND METHODS FOR ASSET DERIVATION FROM GENOMIC SEQUENCES

(71) Applicant: Symphony Diagnostics, Inc., Stateline, NV (US)

(72) Inventors: Julius Barsi, Stateline, NV (US); Arnulf Graf, Stateline, NV (US); Joe Schroeter, Stateline, NV (US)

(21) Appl. No.: 19/330,526

(22) Filed: Sep. 16, 2025

Related U.S. Application Data
(60) Provisional application No. 63/695,103, filed on Sep. 16, 2024.

(51) Int. Cl. G16H 50/20; G16B 30/00; G16B 40/20; G16H 20/10; G16H 50/80

(52) U.S. Cl. CPC G16H 50/20; G16B 30/00; G16B 40/20; G16H 20/10; G16H 50/80

(57) ABSTRACT

Provided herein are systems and methods for selecting sequences that satisfy intent-specific criteria. In particular, provided herein are systems and methods which analyze sequences based on metadata and known, inferred, and experimentally determined attributes to select sequences for desired utilities (e.g., suitable for therapeutic/prophylactic interventions).

### SEQUENCE LISTING

The text of the computer readable sequence listing filed herewith, titled “43573-202_SEQUENCE_LISTING”, created Sep. 16, 2025, having a file size of 3,666 bytes, is hereby incorporated by reference in its entirety.

### FIELD

Provided herein are systems and methods for selecting sequences that satisfy intent-specific criteria. In particular, provided herein are systems and methods which analyze sequences based on metadata and known, inferred, and experimentally determined attributes to select sequences for desired utilities (e.g., suitable for therapeutic/prophylactic interventions).

### BACKGROUND

Antigen characterization, beyond initial detection, is essential for viral diagnostics and possible treatment development. Ideally, any relevant and potentially useful information should be included in such assessment. Various AI/ML approaches have been used to provide a more holistic approach to antigen characterization through a more sensitive viral diagnostics, prediction of epitopes, and antimicrobial resistance. However, these approaches often lack explainability in light of biological findings, accuracy due to high rate of mutation of many pathogens, as well as accurate characterization due to various approaches to genomic sequencing. Recent advances in design and optimization of multiplex genomic sequencing provide vast amount of data. Yet what is lacking in the field is any computational approaches that can optimally utilize the richness of sequencing methods to inform suitability of subsets of sequences for potential downstream uses such as development of nucleic acid based prophylactic and therapeutic products.

### SUMMARY

Provided herein are systems and methods for the selection of sequences that satisfy intent-specific criteria (e.g., suitable for therapeutic/prophylactic interventions).

In some embodiments, provided herein are computer implemented methods comprising obtaining standardized and/or annotated genomic sequence fragments and sample metadata; associating each genomic sequence fragment with one or more attributes; and selecting sequences in which the one or more attributes and/or sample metadata fulfill intent-specific criteria.

In some embodiments, the genomic sequence fragments are derived from a non-human animal sample.

In some embodiments, the genomic sequence fragments are from one or more microorganisms and/or viruses. In some embodiments, the one or more microorganisms and/or viruses comprises any combination of viruses, bacteria, protozoa, algae, and fungi. In some embodiments, the one or more microorganisms and/or viruses comprise one or more pathogenic microorganisms and/or viruses. In some embodiments, the one or more microorganisms and/or viruses comprise one or more non-pathogenic microorganisms and/or viruses. In some embodiments, the one or more non-pathogenic microorganisms comprises one or more endogenous symbiotic microorganisms. In some embodiments, the one or more endogenous symbiotic microorganisms comprises one or more gut flora microorganisms. In some embodiments, the one or more microorganisms and/or viruses comprises an emerging microorganism and/or virus.

In some embodiments, the methods comprise host assessment, or a combined pathogen and host assessment. A host assessment may include, but is not limited to, analyzing host genomic DNA, RNA (including transcriptomic data). This can be used to determine host genetic traits, gene expression profiles, immune response status, disease susceptibility, and/or other physiological states. In some embodiments, one or more inferences on the immune response of the host is made based on host assessment and/or pathogen assessment.

In some embodiments, the sample metadata comprises demographic information, health information, environmental information, or any combination thereof.

In some embodiments, the attributes are associated with each genomic sequence fragment based on data from known databases, inferred or predicted attributes, or contemporary analysis. In some embodiments, the attributes comprise one or more of: level of uniqueness as compared to other sequences or genomes, source organism, list of organisms or species which contain sequence, environment in which sequence was obtained, identification of sequence motifs contained within the genomic sequence fragment, and fitness for prophylactic or therapeutic use.

In some embodiments, the intent is a therapeutic or prophylactic treatment. In some embodiments, the intent comprises development and/or identification of vaccines, antisense oligonucleotides (ASOs), aptamers, reporter genes, natural antagonists to combat pathogens, cis-acting elements, cis-regulatory elements, operons, tertiary structures, organelle targeting sequences, mRNA circularization elements, synthetic barcodes, drug tolerance/resistance genes, GMO signatures, transposon landing sites, regulatory non-coding RNAs, or a combination thereof.

In some embodiments, selecting sequences comprises analyzing the uniqueness of the sequence, desirability for the intent, and suitability of the intent based on the one or more attributes and sample metadata.

In some embodiments, any one or more or all of steps a), b), or c) utilizes an artificial intelligence and machine learning (AI/ML) system.

Also provides are systems comprising a processor running software configured to carry out any or all steps of the methods described herein. In some embodiments, the system is configured to carry out each of the steps. In some embodiments, the system is configured to carry out each of the steps concomitantly and in real-time.

In some embodiments, the system further comprises a sample processing component. In some embodiments, the system further comprises a sample analysis component. In some embodiments, the sample analysis component comprises an automated nucleic acid sequencing component.

Other aspects and embodiments of the disclosure will be apparent in light of the following detailed description and accompanying figures.

### BRIEF DESCRIPTION OF FIGURES

FIG. 1 shows an exemplary flowchart of embodiments of the systems and methods described herein. Raw sequences are imported with sample metadata and automatically: Ingested (Module 1), QCed and Homogenized (Module 2), Fragmented via agnostic, size-independent, reiterative fragmentation of linear sequence (Module 3), and finally, in Module 4A, these sequences are associated with attributes to define the state to which the sequence belongs. In Module 4B, the results are automatically interpreted by aid of ML. Modules 5-7 is a diagnostic panel, which continuously supplies new sequences, reflecting the present pathogen burden. Modules A-Z, derives contemporary real-time assets based on the sequences and associated metadata and attributes compared to intent-based criteria.

FIG. 2 shows an embodiment of the attributes are those properties or characteristics of a sequence considered by the systems and methods described herein. These attributes are used in assessing if the sequence is of interest as an asset, given specific intent-based criteria.

FIG. 3 shows as schematic of an exemplary analysis of a new Listeria sequence of interest using the systems and methods described herein.

FIG. 4 shows a schematic of an exemplary analysis of a tetracycline resistance ribosomal protection protein Tet(M) recently discovered in Clostridioides difficile using the systems and methods described herein.

### DETAILED DESCRIPTION

Designing novel oligonucleotides based on field reports of emerging infectious diseases is important for surveillance and countermeasure (e.g., prophylactics/therapeutics) discovery and development. The disclosed methods and systems identify individual sequences or combinations thereof (whether or not they belong to the infectious agent) that serve as a biomarker and leverage machine learning throughout various stages of this process, including selection of sequences that satisfy the criteria of sequence amenable to therapeutic/prophylactic interventions.

Finding sequences amenable to therapeutic or prophylactic use has, for the most part, followed a list-based approach that focuses on known pathogens and biotoxins. Pathogen outbreaks outside of this list are not readily amenable for intervention, except in infrequent cases where a known therapeutic displays cross-reactivity towards an emerging pathogen. However, these list-based approaches suffer from deficiencies in detecting emerging pathogen variants and designing therapeutics/prophylactics on demand. Overall, these approaches are not amenable to fast turnaround needed to effectively respond to outbreaks.

Non-standard approaches born out of the emergence of a succession of unexpected infectious threats removed the need to look for a specific signature know from previous pathogens and biotoxins. One approach has been to focus not on the pathogen but on host pathways of disease to recommend treatment. However, host-based approaches limit the ability to identify and distinguish individual pathogens, removing any possibility of specific targeting. Other approaches utilize sequencing data to function as a metagenomic classifier for analysis of the pathogen and potential therapeutic or prophylactic options or to describe functions related to microbial pathogenesis encoded in viral and bacterial sequences (but not fungal or protozoa), termed Functions of Sequences of Concern (FunSoCs). These sequences approaches have been limited to single species or single pathogen approaches.

The systems and methods described herein offer advantages over the standard list-based approaches and the more recent non-standard approaches of selecting sequences suitable for use in therapeutic/prophylactic interventions. The systems and methods described herein use the principle of guilt-by association to think of the environment of the pathogen, which includes the host but is not focused on or limited to the host, to annotate a sequence with metadata comprising data and information from other (pathogenic or commensal) microorganisms within the environment into the analysis. Furthermore, the present systems and methods find use for any combinations of pathogens rather than focusing on a single pathogen. Leveraging machine learning throughout various stages of the disclosed systems and methods facilitates automatic result interpretation rather than user-interpreted result generation. Importantly, the disclosed systems and methods facilitate combining detection of the pathogen with derivation of suitability for therapeutic/prophylactic interventions in real-time and on-demand, looking only at those sequences annotated to identify that they are of interest, not limited to previously identified sequences and/or pathogens.

The disclosed systems and methods provide a generalizable process that can be applied to any sequence-based therapeutic/prophylactic. Whereas other systems and methods focus on suitability of a sequence for a sequence-based therapeutic/prophylactic, the disclosed systems and methods further interrogate desirability and uniqueness to discover new sequences from existing and emerging pathogens without comprising sensitivity or specificity. Thus, the disclosed systems and methods are particularly useful in local emerging outbreaks (e.g., in assisting small-to medium-scale environments) without reliance on sporadic, slow-to-arrive and unreliable information from public sources.

The disclosed systems and method overcome challenges found with existing system. These include addressing class-imbalance, for example, where rare events (mutations, phenotypes, adverse outcomes) are underrepresented; high-dimensionality, for example, where data may contains thousands of candidate features and where only a fraction of carry relevant biological signal; and static availability, for example, where datasets are collected in controlled studies or assays and are not continuously refreshed. Conventional classifiers may be inadequate because oversampling introduces artifacts, class-weighting requires sensitive parameterization, and single models tend to bias heavily toward majority classes.

Embodiments of the systems and methods provided herein contribute a “balanced bagging ensemble framework” designed to improve classification performance on imbalanced biological datasets. The system and method comprises one or more or all of the following: 1) data input where offline datasets are divided into pre-defined training and test partitions; feature representation is generated using a bag-of-words-like encoding adapted for biological data (e.g., nucleotide motifs, amino acid subsequences, biomarker identifiers, and/or coded clinical events); 2) balanced subset generation where construction of multiple random balanced subsets is conducted by under-sampling the majority class to match minority class counts; randomization is repeated across iterations to ensure diverse views of the majority class are represented; 3) ensemble training where base classifiers (e.g., decision trees, logistic regression, SVMs) trained independently on each balanced subset; each learner is exposed to equal class proportions, mitigating bias from the original imbalance; 4) decision aggregation where predictions are combined via majority voting, probability averaging, or weighted consensus mechanisms; aggregation enhances robustness and improves minority-class recall without sacrificing specificity; and 5) validation where evaluation is conducted against the held-out test set using metrics appropriate to biotech applications, including ROC-AUC, precision, recall, F1 score, and Matthews correlation coefficient; emphasis is placed on sensitivity to rare but biologically significant classes.

In some embodiments, the analytics component provides several technical advantages when applied to biological datasets, addressing limitations of conventional classifiers in imbalanced, high-dimensional, and offline data settings: 1) no synthetic oversampling, which preserves biological data integrity; 2) bias reduction, since every learner is trained on balanced subsets; classifier diversity, as random under-sampling produces heterogeneous base models; 4) cross-domain applicability, making the approach adaptable to genomics, proteomics, and clinical trial data, among others; 5) optimized for offline datasets, ensuring suitability for assay and study outputs where retraining is costly; and 6) interpretability, because bag-of-words feature encoding provides transparent mappings between biological inputs and classification outputs.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease (e.g., bacterial or viral infection). A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

As used herein, the term “sample metadata” refers to data associated with a sample that provides information about the sample, but that is not determined from experimental analysis of the sample. For a sample obtained from an animal, sample metadata includes, but is not limited to: location of the animal, food source of the animal, health concurrent with or prior to the acquisition of a sample from the animal, reproductive status or history, the nature or identity of kits, assays, techniques, instrument, or reagents used to analyze samples, and the like.

As used herein, the term “computer” refers to a machine, apparatus, or device that is capable of accepting and performing logic operations from software code. The term “application,” “software,” “software code,” or “computer software” refers to any set of instructions operable to cause a computer to perform an operation. Software code may be operated on by a “rules engine” or “processor.” Thus, in some embodiments, the methods and systems of the present invention may be performed by a computer or computing device having a processor based on instructions received by computer applications and software.

The term “electronic computer device” as used herein, is a type of computer comprising circuitry and configured to generally perform functions such as recording and analyzing data; generating, formatting, and analyzing databases; generating reports; storing, retrieving, or manipulation of electronic data; providing electrical communications and network connectivity; or any other similar function. Non-limiting examples of electronic devices include: personal computers (PCs), workstations, laptops, tablet PCs including the iPad, cell phones including iOS phones made by Apple Inc., Android OS phones, Microsoft OS phones, Blackberry phones, digital music players, or any electronic device capable of running computer software and displaying information to a user, memory cards, other memory storage devices, digital cameras, external battery packs, external charging devices, and the like. Certain types of electronic devices which are portable and easily carried by a person from one location to another may sometimes be referred to as a “portable electronic device” or “portable device”.

The term “computer readable medium” as used herein refers to any medium that participates in providing instructions to the processor for execution. A computer readable medium may take many forms, including but not limited to, non-volatile media, volatile media, and transmission media. Non-volatile media includes, for example, optical, magnetic disks, and magneto-optical disks, such as the hard disk or the removable media drive. Volatile media includes dynamic memory, such as the main memory. Transmission media includes coaxial cables, copper wire and fiber optics, including the wires that make up the bus. Transmission media may also take the form of acoustic or light waves, such as those generated during radio wave and infrared data communications. Non-transitory computer readable media includes all computer readable media, with the sole exception being a transitory, propagating signal per se.

As used herein the term “data network” or “network” shall mean an infrastructure capable of connecting two or more computers such as client devices either using wires or wirelessly allowing them to transmit and receive data. Non-limiting examples of data networks may include the Internet or wireless networks which may include Wi-Fi and cellular networks. For example, a network may include a local area network (LAN), a wide area network (WAN) (e.g., the Internet), a mobile relay network, a metropolitan area network (MAN), an ad hoc network, a telephone network (e.g., a Public Switched Telephone Network (PSTN)), a cellular network, a Zigby network, or a voice-over-IP (VOIP) network.

As used herein, the term “database” shall generally mean a digital collection of data or information. For the purposes of the present disclosure, a database may be stored on a remote server and accessed by a client device (e.g., through the Internet) or alternatively in some embodiments the database may be stored on the client device or remote computer itself.

As used herein, the term “artificial intelligence” shall generally mean smart machines capable of performing tasks that typically require human-like intelligence and the machines learning from experience, adjusting to new inputs, processing large amounts of data, and recognizing patterns in the data.

As used herein, the term “machine learning” shall generally mean smart machines using statistics to find patterns in large amounts of data, wherein the data is anything that can be digitally stored. Machine learning is seen as a subset of artificial intelligence, and machine learning algorithms make predictions based on data without being programmed to specifically do so.

As used herein, the term “deep learning” is a subset of machine learning that uses artificial neural networks with a large number of hidden layers. Such networks were designed to simulate brain-like processing of complex information, for example, to progressively extract higher level features from raw data input. These networks can comprise convolutional as well as recurrent networks.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids, such as DNA and RNA, are found in the state they exist in nature. Examples of non-isolated nucleic acids include a given DNA sequence (e.g., a gene) found on the host cell chromosome in proximity to neighboring genes; RNA sequences, such as a specific mRNA sequence encoding a specific protein, found in the cell as a mixture with numerous other mRNAs which encode a multitude of proteins. However, isolated nucleic acid encoding a particular protein includes, by way of example, such nucleic acid in cells ordinarily expressing the protein, where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide will contain at a minimum the sense or coding strand (i.e., the oligonucleotide may be single-stranded), but may contain both the sense and anti-sense strands (i.e., the oligonucleotide may be double-stranded). An isolated nucleic acid may, after isolation from its natural or typical environment, be combined with other nucleic acids or molecules. For example, an isolated nucleic acid may be present in a host cell into which it has been placed, e.g., for heterologous expression.

The term “purified” refers to molecules, either nucleic acid or amino acid sequences that are removed from their natural environment, isolated, or separated. An “isolated nucleic acid sequence” may therefore be a purified nucleic acid sequence. “Substantially purified” molecules are at least 60% free, preferably at least 75% free, and more preferably at least 90% free from other components with which they are naturally associated. As used herein, the terms “purified” or “to purify” also refer to the removal of contaminants from a sample. The removal of contaminating proteins results in an increase in the percent of polypeptide or nucleic acid of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contain a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The present disclosure provides automated systems and methods related to asset derivation for genomic sequences. In particular, the present disclosure provides systems and methods to obtain (e.g., collect, prepare, input, process, and store) sequences with metadata and attributes associated with the sequences and select sequences based upon the associated metadata and attributes for specific criteria for a downstream value or potential intent or use.

As provided herein, the methods find use to evaluate the new and known genomic sequences to determine potential value of any particular sequence, for example, vaccine development, antisense oligonucleotides (ASOs), aptamers (e.g., for detection or therapy), reporter genes (e.g., for in vivo cargo delivery and/or biodistribution), natural antagonists to combat pathogens, cis-acting elements, cis-regulatory elements, operons, tertiary structures, organelle targeting, mRNA circularization elements, synthetic barcoding, drug tolerance/resistance, GMO signature identification, transposon landing sites, and/or regulatory non-coding RNAs.

The methods and systems include obtaining standardized and/or annotated genomic sequence fragments and sample metadata; associating each genomic sequence fragment with one or more attributes and selecting sequences in which the one or more attributes and/or sample metadata fulfill intent-specific criteria.

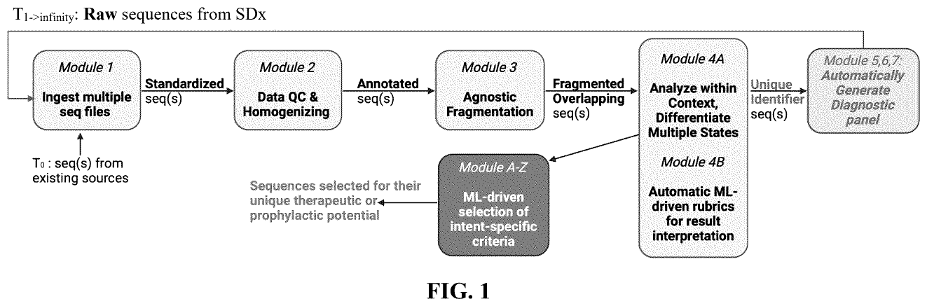

Obtaining standardized and/or annotated genomic sequence fragments and sample metadata can include information about the sample which provided the sequence. As shown in FIG. 1, raw sequences can be obtained with sample metadata (individual and population status) and automatically ingested (Module 1), subjected to quality control and homogenized (Module 2), and fragmented via agnostic, size-independent, reiterative fragmentation of linear sequence (Module 3).

In some embodiments, obtaining standardized and/or annotated genomic sequence fragments is automated. In some embodiments, obtaining standardized and/or annotated genomic sequence fragments comprises i) isolating nucleic acid from a sample; and ii) sequencing the isolated nucleic acid. In some embodiments, the method further comprises amplifying the nucleic acid prior to the sequencing.

In some embodiments, the system comprises an AI/ML component previously trained on sample metadata and sequence signatures to associate a sequence with sample metadata. In some embodiments, the sample metadata and standardized and/or annotated genomic sequence fragments are from one or more previously analyzed samples.

Sample metadata includes individual or population sources for the sequences. For example, the metadata may include information about what the situation was that identified the sequence(s), e.g., the macroscopic environment (e.g., type of host, environment of host, other non-host sequences present in sample, etc.) in which the sample that contains the sequence was obtained. The metadata may additionally include information regarding whether the sequence(s) was present in samples classified as highly pathogenic/infective or found in presence of other sequences within the same sample that belong to a different species or composition.

In some embodiments, the sample metadata comprises demographic information. In some embodiments, the demographic information comprises age, birth date, number of siblings, gender, species, sub-species, breed, coloring, weight, birth weight, height, and length. In some embodiments, the sample metadata comprises health information. In some embodiments, the health information comprises: disease history, vaccination status, medication history, antibiotic history, pregnancy history, allergies, injury history, behavioral abnormalities, medical test history, medical procedure history, diet (e.g., food ingested), nutritional supplement history, and growth history. In some embodiments, the sample metadata comprises environmental information. In some embodiments, the environmental information comprises: present geography, historical geography, air quality, water quality, soil quality, presence of same-species animals, density of same-species animals, presence of different-species animals, weather, exposure to disease vectors, proximity to disease vectors, exposure to radiation, time spent outdoors, time spent indoors, feeding conditions (e.g., food consumed), geographic history, forest coverage, fertilizer exposure, sewage conditions, exposure to emissions (e.g., sulfur dioxide), and slaughter conditions.

The sequences may be gathered from a variety of samples. In some embodiments, the sample is a non-human sample. For example, the sample may be from a non-human animal (e.g., a farm animal, a companion animal, a wild animal, an aquatic animal, an animal in captivity, an endangered or threatened species of animal). In some embodiments, the sample is from a human.

In some embodiments, the sample is from a farm animal. In some embodiments, the farm animal is selected from the group consisting of dairy cattle, sheep, horses, goats, chickens, pigs, rabbits, deer, turkeys, mules, banteng, boars, bison, beef cattle, emu, donkeys, geese, camels, reindeer, pheasants, ducks, quails, domestic yaks, llamas, American pygmies, alpacas, ostrich, elk, and fish. In some embodiments, the animal is a companion animal. In some embodiments, the companion animal is a dog, cat, horse, rabbit, ferret, bird, guinea pig, fish turtle, snake, or lizard. In some embodiments, the animal is a wild animal. In some embodiments, the wild animal is a lion, tiger, leopard, cheetah, jaguar, elephant, giraffe, hippopotamus, rhinoceros, gorilla, chimpanzee, orangutan, bear, wolf, coyote, fox, lynx, bobcat, mountain lion, zebra, wildebeest, gazelle, antelope, warthog, hyena, jackal, crocodile, alligator, turtle, snake, kangaroo, koala, wombat, wallaby, platypus, octopus, squid, crab, lobster, shrimp, clam, oyster, snail, walrus, seal, whale, dolphin, manatee, skink, lizard, gecko, chameleon, bat, raccoon, opossum, rat, mouse, chipmunk, rabbit, badger, skunk, armadillo, porcupine, beaver, otter, seagull, eagle, flacon, hawk, osprey, vulture, owl, parrot, heron, swan, goose, duck, ostrich, turkey, emu, camel, llama, yak, deer, moose, caribou, bison, buffalo, or elk. In some embodiments, the animal is an aquatic animal. In some embodiments, the aquatic animal is a carp, pollock, clam, tilapia, shrimp, tuna, anchovy, salmon, herring, mackerel, rohu, cod, squid, trout, crab, sardine, haddock, catfish, eel, scallop, prawn, shark, perch, albacore, or bass. In some embodiments, the animal is an endangered or threatened species.

In some embodiments, the sample is derived from a human or non-human animal having or suspected of having a disease or disorder. In some embodiments, the sample is derived from a human or non-human animal having or suspected of having a disease or disorder mediated by one or more microorganisms or viruses. In some embodiments, the sample is derived from a healthy human or non-human animal.

In some embodiments, the genomic sequence fragments are from present in the sample. Microorganisms include bacteria, protozoa, algae, and fungi. In some embodiments, the one or more microorganisms and/or viruses comprises any combination of viruses, bacteria, protozoa, algae, and fungi.

In some embodiments, the one or more microorganisms and/or viruses comprise one or more pathogenic microorganisms and/or viruses. In some embodiments, the one or more microorganisms and/or viruses comprises one or more non-pathogenic microorganisms and/or viruses. In some embodiments, the one or more non-pathogenic microorganisms comprises one or more endogenous symbiotic microorganisms. In some embodiments, the one or more endogenous symbiotic microorganisms comprises one or more gut flora microorganisms.

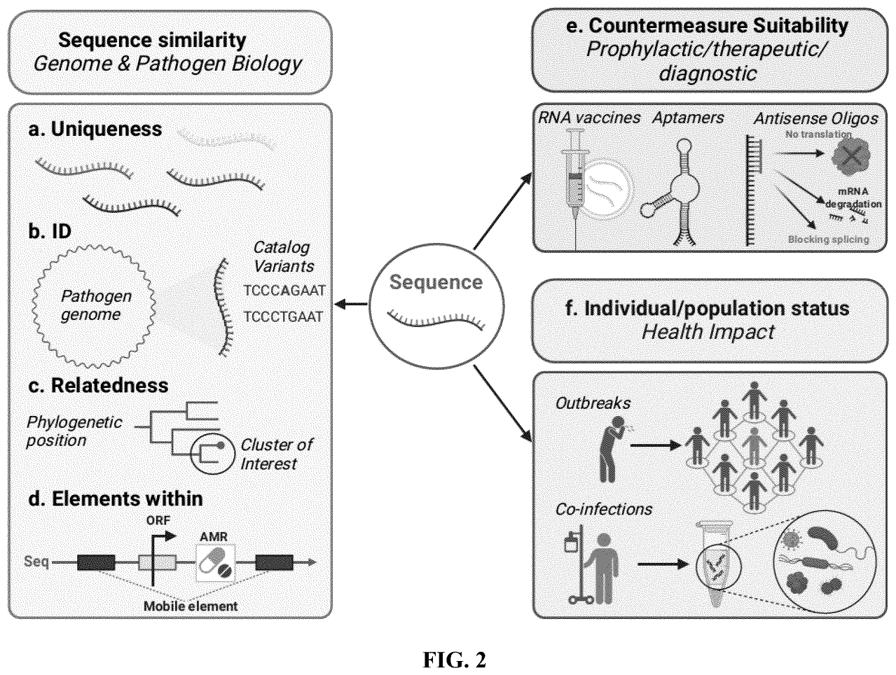

The sequences and their metadata are associated in the context of one or more attributes. This is outlined in Modules 4A/4B in the exemplary flow chart of FIG. 1. The attributes are those properties or characteristics of a sequence that have broad impacts on utility. In some embodiments the attributes include, but are not limited to, level of uniqueness as compared to other sequences or genomes, source organism, list of organisms or species which contain sequence, environment in which sequence was obtained, identification of sequence motifs contained within the genomic sequence fragment, and suitability for prophylactic or therapeutic use (FIG. 2).

In some embodiments, associating each genomic sequence fragment with one or more attributes is automated. In some embodiments, the system comprises an AI/ML component previously trained on attributes and a library of sequences to associate each genomic sequence fragment with one or more attributes.

In some embodiments, the one or more attributes comprise sequence similarity. Sequence similarity reflects the biology of the sequence and the genome(s) which comprise the sequence. Sequence similarity provides a measure of the uniqueness of the sequence in the context of all other sequences and/or genomes. For example, the sequence similarity may be a measure of how dissimilar the sequence is compared to all other sequences.

In some embodiments, the one or more attributes comprise what organism(s) comprises or contains the sequence or highly related similar sequences. For example, the attribute may provide a list or identity of the organism or group of organisms from which the sequence most likely originated and annotation of the differences (sequence variants) to that sequence. For example, the attributes can provide context for the phylogenetic position of the species/sub-species/variant the sequence belongs to.

In some embodiments, the one or more attributes comprise the biological features of the sequence. For example, the one or more attributes include the motifs within the sequence that are informative of its genome biology (regulatory, splicing, coding, etc.), function (e.g., antimicrobial resistance), or history (transposed sequence, plasmid features, etc.), see Table 1. Thus, the sequence can be annotated with known and putative elements.

TABLE 1
Exemplary motifs within a sequence

| Information | | |
|---|---|---|
| content | Category | Examples |
| Genome | Open Reading | Protein-coding genes, Peptide- |
| Biology | Frames (ORFs) | encoding ORFs, LncRNAs, small |
| | | RNAs, Frame-shift super-encoded |
| | | ORFs |
| | Cis-regulatory | Transcription factor binding sites, |
| | elements | Promoters, IRES (Internal Ribosomal |
| | | Entry Sites) |
| | Splicing elements | Splice junctions and variants |
| Function | Functional | Antimicrobial resistance, Drug |
| | elements | susceptibility loci, Immune- |
| | | subverting loci |
| History | Transposition | Mobile elements, Inverted repeats |
| | elements | |
| | Elements of recent | Horizontal gene transfer elements, |
| | acquisition | Plasmid features |
| | Polymorphism | Within-genome variants |

In some embodiments, the one or more attributes comprise information reflecting if the sequence is suitable for prophylactic/therapeutic purposes. For example, the sequence can be annotated to reflect regions compatible with prophylactic/therapeutic modalities that utilize sequences either in their mode of action or a starting point for prophylactic/therapeutic design. Examples of such modalities are in Table 2.

TABLE 2
Exemplary nucleic acid therapeutics

| Class | Examples |
|---|---|
| RNA vaccines | Multi-antigen self-amplifying RNA |
| | vaccines |
| Antisense Oligos | Locked Nucleic Acid probes (LNAs), |
| (ASO) | Splice-blockers |
| Aptamers | DNA, RNA, peptides, conjugates |
| Other | Acoustic Reporter Genes (Sound-based |
| | Pathogen imaging) |

The one or more attributes may include or utilize data and information from multiple sources. For example, the source of information used to associate one or more attributes to a sequence can be gathered from databases of publicly known and curated data, a machine learning based model which outputs inferences and predictions (e.g., likely attributes that are not directly observed) based on the data/metadata associated with the sequence, and contemporary data acquired in real-time (e.g., when an emergent pathogen is identified). See Table 3.

In some embodiments, new data and sequences are acquired by a diagnostic panel, which can continuously supply new sequences for collecting, preparing, inputting, processing, and storing as well as for use in the machine learning predictions and inferences.

In some embodiments, the predictions are performed by an AI/ML system automatically performing multidimensional analytics.

TABLE 3
Sources of attributes

| Source | Type of Source | Associated Attribute |
|---|---|---|
| Known Databases | Genome & | Comparisons to known sequences |
| | Sequence | uniqueness |
| | databases | source organism |
| | | differences to known genome sequences |
| | | closely related organisms |
| | | motifs |
| | Sequence-to- | Functional motifs |
| | Function | Plasmid features |
| | databases | Experimentally derived functional data (e.g., |
| | | protein interactions) |
| Inferred/Predicted | Inferences | Bookmark top unique sequences |
| | | Assign variants if there are differences between |
| | | the pathogen genome and sequence |
| | | Assign pathogenicity based on relatives of interest |
| | | Assign all possible biological functions, identify |
| | | origin |
| | Machine | Check uniqueness against incoming new sequences |
| | Learning | Predict pathogen ID if unclear |
| | Predictions | Predict pathogenicity/threat level based on the |
| | | pathogen: phylogenetic position and collective |
| | | biological features of all sequences that belong |
| | | to it. |
| | | Predict if co-infections (simultaneous presence of |
| | | 2 and more pathogens) represent a threat. Assess |
| | | “guilt-by-association” - the presence of |
| | | underlying pathogen signature, reflected in the |
| | | composition of the sample microbial community. |
| | | Automatically derive rubrics by which to interpret |
| | | assay results. |
| Contemporary | Pathogen | De novo sequences of new variants |
| Assay Data | sequence | |
| | Sample | Outbreak monitoring |
| | metadata | |
| | Sequences | Catalog if variants of the same genome are present |
| | within sample | Catalog possible co-infections |

Following the association of a sequence with the one or more attributes (e.g., collected, associated, retained, inferred, and predicted attributes as described above), the sequences can then be selected for by intent-specific criteria (e.g., for a particular therapeutic/prophylactic potential) which align with the one or more attributes of the sequence.

The intent can comprise any potential downstream application of the sequences. In some embodiments, the intent comprises a therapeutic or prophylactic for which the sequence may be suitable. For example, the intent includes suitability of use of the sequence for: vaccine development, antisense oligonucleotides (ASOs), aptamers (e.g., for detection or therapy), reporter genes (e.g., for in vivo cargo delivery and/or biodistribution), natural antagonists to combat pathogens, cis-acting elements, cis-regulatory elements, operons, tertiary structures, organelle targeting, mRNA circularization elements, synthetic barcoding, drug tolerance/resistance, GMO signature identification (e.g., poly-linkers, FLIP sites, CRE sites, commercial plasmid signature, DNA insertion “scars” in the form of palindromic flanking sequences), transposon landing sites, multidrug resistance proteins (MRPs), and regulatory non-coding RNAs.

The selection of criteria takes into account the uniqueness of the sequence, desirability and suitability. Desirability is related to how important it is to fill the intent. In some embodiments, the desirability is connected to the sample metadata, e.g., how infectious is this new variant (e.g., highly infectious, medium, low or unknown), how difficult is it to treat (e.g., antibiotic resistance), and where/when was it detected (e.g., temporal & spatial information). The temporal and spatial information adds to the desirability as a measure of urgent it is to fulfill the intent. Suitability is related to what characteristics makes a good agent for the intent and includes applying the attributes and metadata as described above. Lastly the uniqueness applies the specificity by examining the ability of a selected sequence to perform the intent as compared to all other sequences. Table 4 shows an exemplary set of characteristics and specific measures of desirability and suitability for different nucleic acid based intents.

TABLE 4
Selection by intent-specific criteria

| | | Criteria | |
|---|---|---|---|
| Intent | Characteristics of Sequence | Desirability | Suitability |
| Find sequences of import for | 1. Located within a coding | Sequence belongs | Similarity to an |
| mRNA vaccine | region | to an emerging | experimentally-derived |
| development (prevention) | 2. Encodes for a unique | pathogen with high | peptide epitope or |
| (Example 1) | cell-surface peptide | infectivity and low | otherwise singled out by |
| | | chance of | ML algorithm. |
| | | containment. | |
| Antisense Oligos (ASO) to | 1. DNA codes for mRNA | Sequence belongs | If bound by ASO, it will |
| suppress translation, alter | sequence | to a treatment- | affect pre-mRNA |
| splicing or otherwise disrupt | 2. The product of the | resistant pathogen. | maturation (polyA, |
| pathogen-variant-specific | mRNA is crucial to | | splicing, cleavage) or |
| mRNAs (treatment) | pathogen function | | mRNA translation |
| (Example 2) | | | (inhibition, cleavage, |
| | | | turnover rate). |
| Aptamers for detection | Sequence with particular | 1. Aptamers that | +Signaling Aptamers |
| (e.g., instrument-free | binding constant & Gibbs | operate on kinetic | +Protease Inhibitory |
| diagnostic tests), or therapy | free energy when bound to | competition with | Aptamers |
| (targeting pathogen | target. | ligand. | +Electrical detection by |
| metabolism via small | | or | way of aptamers |
| molecules) | | 2. Aptamers that | endowed with nanogap |
| | | operate on | break-junctions |
| | | equilibrium | +Aptamers as |
| | | competition with | instruments for super- |
| | | ligand | resolution microscopy |
| | | | +Nanotextured |
| | | | substrates decorated with |
| | | | immobilized aptamers |
| Acoustic Reporter Genes | Sequence that encodes | Heterologous | Genetic constructs that |
| for deep tissue visualization | gas-filled protein | expression of | enable gene expression |
| of in vivo cargo delivery | nanostructures | engineered gene | to be visualized in vivo, |
| and/or biodistribution | | clusters encoding | through deep tissue, by |
| (mRNA vaccines) | | gas vesicles | way of ultrasound |
| Usurp natural antagonists | Sequence that encodes an | Ability to properly | Each phage has evolved |
| to combat pathogens. | Endolysin, (peptidoglycan | fold quaternary | specificity towards |
| Pathogen-specific | hydrolase) to | structure when | bacterial species down to |
| bacteriophages as a means | enzymatically digest the | overexpressed in a | the level of bacterial |
| to control bacteria | peptidoglycan layer of the | foreign | strains. |
| | target bacterium. | cell/environment. | |
| Viral cis-acting elements to | Such sequences are notoriously difficult to pinpoint due, in part, | | |
| manipulate the activity of | to the fact that many viruses encode distinct proteins within | | |
| essential viral genes | the same sequence by exploiting a frameshift. | | |
| Eukaryotic cis-regulatory | A collection of sequence | Capable of | Ability to Activate, |
| elements to manipulate | motifs that are | integrating multiple | Repress, or otherwise |
| gene activity | functionally modular and | “trans” information | modulate gene |
| | serve as DNA binding | to execute AND; | expression in a highly |
| | sites for transcription | OR; NOR logic | specific manner. |
| | factors | | |
| Prokaryotic operons to | Combination of regulatory | Self-contained | Construction of genetic |
| manipulate gene activity | sequence and functionally | multi-component | circuits with integrated |
| | related genes under its | gene expression | feedback loops and other |
| | immediate control | system | complex design |
| | | | principles in vivo |
| Functional genomic | Ability to facilitate | Usurp cellular | +Coordinating parallel |
| tertiary structures (e.g., | ribosome entry without | physiology for un- | expression of multiple |
| IRES) to manipulate cellular | scanning, or attract other | natural purposes | genes with the same |
| physiology | components of the cellular | that have been | vector |
| | machinery by by-passing | artificially designed | +To target elements that |
| | cellular quality control | | are crucial for viral |
| | | | circumvention of natural |
| | | | defense mechanisms. |
| Organelle targeting (e.g., | In much the same way that embedding NLS within the molecule | | |
| nuclear localization signal | ensures that the molecule is delivered into the nucleus, | | |
| (NLS)) to deliver small | a similar strategy can target the Golgi, plasma membrane, | | |
| molecules by usurping | ER, mitochondria, etc. | | |
| innate cellular mechanisms | | | |
| mRNA circularization | poly A length and | Ability to attract | Control mRNA half-life, |
| elements | associated attributes | key co-factors | turnover, stability |
| | | necessary for | |
| | | translation | |
| Synthetic barcoding | Orthogonal sequence | Serve as a Unique | Massively parallel |
| | designed entirely in a lab | Molecular | reporter assays to |
| | and never to be found in | Identifier for a | monitor transgene |
| | nature. | variety of | biodistribution via |
| | | oligonucleotides. | sequencing. |
| Drug tolerance/resistance | Sequence encodes a | Belongs to the | Predict effectiveness |
| | diverse repertoire of ATP- | multidrug | of contemporary |
| | binding cassette (ABC) | resistance protein | countermeasures |
| | transporters | (MRP) family | |
| Vaccine “escape velocity” | Identifying allelic variants | Ability to record | Predict effectiveness of |
| tracking | that disrupt a known | and model | contemporary vaccines |
| | epitope exploited by an | mutational trends of | |
| | existing vaccine | therapeutic targets | |
| GMO signature | multiple cloning site | Distinct signature | finding evidence |
| identification | (MCS), also called a poly- | indicative of | indicative of genetic |
| | linker, FLIP sites, CRE | laboratory design. | manipulation |
| | sites, commercial plasmid | | |
| | signature, DNA insertion | | |
| | “scars” in the form of | | |
| | palindromic flanking | | |
| | sequences . . . | | |
| Retro-homing transposon | Mobile Bacterial Group II | Self-splicing | Targetrons, RNA-guided |
| landing sites | Introns | Retrotransposons | gene targeting agents for |
| | | | bacterial genome |
| | | | engineering |
| Piwi IncRNAs to fertility | Small non-coding RNAs | Discriminatory | Suppresses transposons |
| implications | | power to discern | across germline cells to |
| | | genuine genes and | ensure fertility, despite |
| | | repress self- | varied genomic assaults |
| | | replicating genetic | |
| | | elements | |

In some embodiments, the systems and methods use supervised and unsupervised ML to score and subsequently rank the sequence attributes and metadata and apply them to the intent specific criteria for uniqueness, suitability, and desirability in regards to the intent as outlined in Table 4. In some embodiments, the systems and methods use ensemble learning to boost accuracy of associating sequence attributes and metadata with a sequence and sequence selection. In some embodiments, the systems and methods further increase prediction accuracy by applying new sequences and their sequence attributes and metadata and utility. In order to leverage intent-specific criteria delineated in Table 4, we employ Generative AI and variants of reinforcement learning to create novel derivatives using our derivative databases (each, a comprehensive collection of experimentally validated genomic elements that are categorized according to biological function). Our creative process of generating new derivatives is based on a careful balance between exploration and exploitation. While this process could be achieved by cross referencing species-specific sequence against particular in-house databases, our proprietary ML models permit us to automatize this process and scale it to larger datasets. Consequently, the catalog of derivatives that are thus identified and the models used to create them, collectively imply that each query sequence linked to one of our proprietary databases is also endowed with the value harbored therein. The output of these derivatives may take the form of either (1) cataloged sequences, (2) genomic coordinates, or (3) a dynamic window that reveals features/value as it scans across sequence space.

Any sequence within a sample will be considered in the disclosed systems and methods for possible selection if it satisfies the intent-specific criteria. Thus, sequence(s) that encompass a whole phylogenetic branch of pathogens of interest can be targeted simultaneously. For example, if the objective is to find a sequence to specifically target the SARS-COV-2 Alpha B.1.1.7 variant, then the selection would be defined to all flavors of Alpha B.1.1.7 variant and all other SARS-COV-2 variants, other coronaviruses, or other pathogen/species would be excluded from the selection. In short, the selection and resulting sequence would be specific to the Alpha B.1.1.7 variant. Similarly, if another objective is to encompass all known SARS-COV-2 variants, the selection would separate all SARS-COV-2 variants from all other coronaviruses, and the selected sequence would be defined to target all of SARS-COV-2 variants.

In some embodiments, a component of the systems and methods are assay kits that facilitate the collection of biological data from samples. In some embodiments, the assay kits comprise multiplex reaction devices (e.g., multi-well plates) and reagents (e.g., a cocktail of oligonucleotides that function as multiplex PCR primer-pairs capable of amplifying a portion nucleic acid from multiple microorganisms or viruses). In some embodiments, samples undergo a processing and pre-purification step prior to nucleic acid amplification. For example, in some embodiments, samples may undergo cellular lysis, dilution, or concentration. In some embodiments, nucleic acid is purified way from non-nucleic acid components of the sample, by, for example, capture, centrifugation, filtration, or the like. Target sequences in the nucleic acid may be amplified any suitable methodology. Illustrative non-limiting examples of nucleic acid amplification techniques include, but are not limited to, polymerase chain reaction (PCR), TAQMAN amplification, reverse transcription polymerase chain reaction (RT-PCR), transcription-mediated amplification (TMA), ligase chain reaction (LCR), strand displacement amplification (SDA), and nucleic acid sequence-based amplification (NASBA). Those of ordinary skill in the art will recognize that certain amplification techniques (e.g., PCR) typically involve RNA reverse transcription to DNA prior to amplification (e.g., RT-PCR), whereas other amplification techniques directly amplify RNA (e.g., TMA and NASBA).

In some embodiments, the assay kit simultaneously targets known microorganisms and viruses, yet has the capability to discover novel viral, bacterial, fungal, and parasitic pathogens of interest. In some embodiments, the assay kit is composed primarily of multi-well plates, with each well harboring a cocktail of oligonucleotides that function as multiplex PCR primer-pairs capable of amplifying a portion of the genome from multiple microorganisms or viruses. These amplicons are then analyzed (e.g., sequenced using long-read sequencing). In some embodiments, a total of 30-50 primer pairs are employed in the assay kit, optimized to show minimal primer-dimer amplification (unwanted amplicons observed in the absence of a template, resulting from starting a PCR reaction off another primer within the primer mix). While the primers are capable of amplifying a subset of the pathogen genome, the sequence variants within the primer-defined genomic region are identified by later analysis (e.g., sequencing). This is especially relevant to the emerging variants of known strains or antimicrobial resistance/toxin-encoding genes.

In some embodiments, a component of the systems and methods is hardware. In some embodiments, the hardware comprises automated sample and liquid handling components that orchestrate processing of collected samples through optional sample pre-purification steps, through assay kit sample processing, and through nucleic acid analysis and data collection. For the latter, in some embodiments, the systems and methods comprise a nucleic acid sequencer that determines target sequences from the amplified nucleic acids generated by the assay kits. Nucleic acid may be analyzed using a variety of techniques including but not limited to: nucleic acid sequencing, nucleic acid hybridization, nucleic acid amplification, and mass spectroscopy. The description herein focuses on sequencing to illustrate embodiments of the invention.

Suitable nucleic acid sequencing techniques include, but are not limited to, sequencing by synthesis (see e.g., Meyer and Kircher, “Illumina sequencing library preparation for highly multiplexed target capture and sequencing,” Cold Spring Harbor Protocols 2010 (6)); single-molecule real-time sequencing (see e.g., Levene et al., “Zero-Mode Waveguides for Single-Molecule Analysis at High Concentrations,” Science. 299 (5607): 682-6 (2003)); ion semiconductor sequencing (see e.g., Rusk, “Torrents of sequence,” Nat. Methods 8, 44 (2011)); pyrosequencing (see e.g., Wicker et al., “454 sequencing put to the test using the complex genome of barley,” BMC Genomics, 7:275, 2006); sequencing by ligation (SOLiD sequencing) (see e.g., Margulies et al., “Genome sequencing in microfabricated high-density picolitre reactors,” Nature, 437:376-80 (2005)); nanopore sequencing (see e.g., Goodwin et al., “Oxford Nanopore sequencing, hybrid error correction, and de novo assembly of a eukaryotic genome,” Genome Res., 25 (11): 1750-6 (2015)); chain termination sequencing (Sanger sequencing) (see e.g., Sanger et al., “DNA sequencing with chain-terminating inhibitors, “Proceedings of the National Academy of Sciences of the United States of America, 74 (12): 5463-5467 (1977)); and sequencing with mass spectrometry (see e.g., Edwards et al., “Mass-spectrometry DNA sequencing,” Mutation Research, 573 (1-2): 3-12 (2005)). The use of Oxford Nanopore Technology allows for the obtaining of >10 kb uninterrupted sequences-enough to resolve whole plasmids (harboring antimicrobial resistance genes) and the phylogenetic relationship of new emerging variants of microorganisms or viruses relative to existing ones circulating through the population.

In some embodiments, robotic sample and liquid handling according to sample input track the progress of each sample. For instance, computer vision is used to optimize the sample and liquid handling of the robot, and track the evolution of each biological sample.

In some embodiments, the technology described herein is associated with a programmable machine designed to perform a sequence of arithmetic or logical operations as provided by the methods described herein. For example, some embodiments of the technology are associated with (e.g., implemented in) computer software and/or computer hardware. In one aspect, the technology relates to a computer comprising a form of memory, an element for performing arithmetic and logical operations, and a processing element (e.g., a microprocessor) for executing a series of instructions (e.g., a method as provided herein) to read, manipulate, and store data.

In some embodiments, the various embodiments of the present disclosure are associated with a plurality of programmable devices that operate in concert to perform a method as described herein. For example, in some embodiments, a plurality of computers (e.g., connected by a network) may work in parallel to collect and process data, e.g., in an implementation of cluster computing or grid computing or some other distributed computer architecture that relies on complete computers (with onboard CPUs, storage, power supplies, network interfaces, etc.) connected to a network (private, public, or the internet) by a conventional network interface, such as Ethernet, fiber optic, or by a wireless network technology.

For example, some embodiments provide a computer that includes a computer-readable medium. The embodiment includes a random access memory (RAM) coupled to a processor. The processor executes computer-executable program instructions stored in memory. Such processors may include a microprocessor, an ASIC, a state machine, or other processor, and can be any of a number of computer processors, such as processors from Intel Corporation of Santa Clara, California and Motorola Corporation of Schaumburg, Illinois. Such processors include, or may be in communication with, media, for example computer-readable media, which stores instructions that, when executed by the processor, cause the processor to perform the steps described herein.

Computers are connected in some embodiments to a network. Computers may also include a number of external or internal devices such as a mouse, a CD-ROM, DVD, a keyboard, a display, or other input or output devices. Examples of computers are personal computers, digital assistants, personal digital assistants, cellular phones, mobile phones, smart phones, pagers, digital tablets, laptop computers, internet appliances, and other processor-based devices. In general, the computers related to aspects of the technology provided herein may be any type of processor-based platform that operates on any operating system, such as Microsoft Windows, Linux, UNIX, Mac OS X, etc., capable of supporting one or more programs comprising the technology provided herein. Some embodiments comprise a personal computer executing other application programs (e.g., applications). The applications can be contained in memory and can include, for example, a word processing application, a spreadsheet application, an email application, an instant messenger application, a presentation application, an Internet browser application, a calendar/organizer application, and any other application capable of being executed by a client device. All such components, computers, and systems described herein as associated with the technology may be logical or virtual.

In some embodiments, the systems and methods employ system control hardware and software that manages the hardware, that processes and analyzes sequences, sample metadata, and attributes, and selects sequences based on intent-specific criteria. In some embodiments, software is run on a computer processor. In some embodiments, the system and system software is multilayered, modular and scalable. In some embodiments, the software manages one or more of the following system/method operations and features: collection of sample metadata associated with each sample; collect, compile and organize all sequences and sample metadata; leverage machine learning for a variety of purposes, including the inference of non-obvious attributes; selection of sequences; and other operations and feature of the disclosed methods. In some embodiments, the systems and methods perform one or more or all of the above concomitantly and in real-time.

An artificial intelligence system component may comprise or function as artificial intelligence logic stored in memory that may be executable by a processor of one or more servers and/or client devices. In some embodiments, the artificial intelligence component may function as or comprise a machine/deep learning/artificial intelligence platform that interrogates the information or data of the system and learns about trends associated with data obtained from analysis of one or more samples, sample metadata, and information in public or private databases. The artificial intelligence component consistently undergoes algorithm testing and validation based on new data available.

### EXAMPLES

### Example 1

### Listeria Epitopes

In this example, the intent-specific criterion is to identify candidates for vaccine development, as illustrated for a newly emerging variant of Listeria. The goal is to identify sequences that facilitate the development of a vaccine intended to protect against this new variant.

A diagnostic panel, designed to capture existing and new Listeria species/variants (among other pathogens), identifies a set of sequences that unequivocally belong to Listeria, according to the assay. Furthermore, within the constraints of the system, novel sequence variants are detected in one or more of the amplicons. A subset of these sequences (n≥1) is identified by Module 4A (FIG. 1) as unique in that it cannot be found in any other species (host or pathogen), including all known Listeria variants and sequences. In addition, these sequences are mapped onto the Listeria genome (e.g., their position within the genome) and annotated with all Motifs of value. For example, at a minimum, the sequences are identified having a coding sequence, a regulatory sequence, and/or having a known function.

Module A is then responsible for matching the sequence with the selection criteria, including desirability and suitability, used to identify candidates for vaccine development.

Desirability is related to how important is it to find a vaccine for this specific variant. The desirability is intertwined with the context in which the variant is identified. Examples of this context associated with the sequence as its metadata are: how infectious is this new variant (e.g., highly infectious, medium, low or unknown), how difficult is it to treat (e.g., antibiotic resistance), and where/when was it detected (e.g., temporal & spatial information).

The temporal value is in knowing that the unique sequence is derived from an emergent variant. The temporal and spatial information adds a measure of urgency. For example, a variant that has been detected using a diagnostic assay relatively recently for the first time, and since then, it has gained a foothold across large geographical regions, is much more valuable for vaccine purposes than a unique sequence from a variant that has been around for a while and is contained. Note that this criterion will change as new data come in (e.g., desirability has a timestamp).

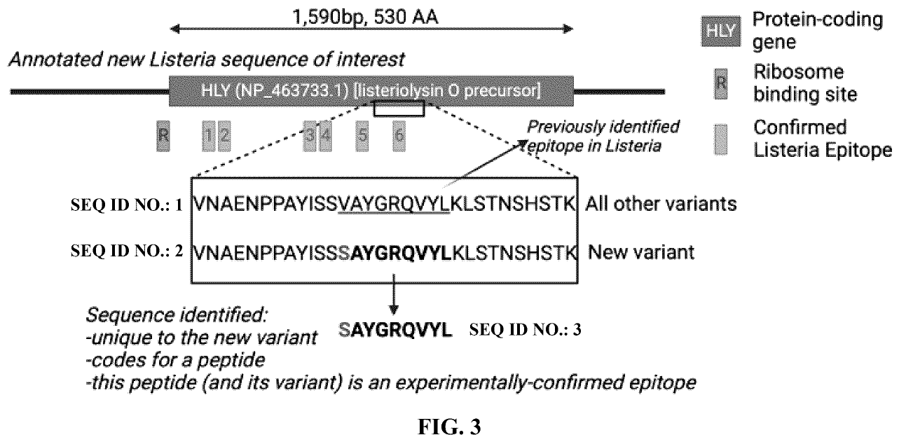

Suitability is related to what assets makes a good vaccine. For suitability Module A communicates with a proprietary epitope database populated by a collection of curated published sources enriched with SDx-collected data. The process is illustrated in FIG. 3. This process (i) selects all Listeria Variant Unique DNA sequences that encode protein sequence, (ii) takes+/−15 amino acids flanking the sequence change (total of 31 AAs peptide), (iii) scans for AA sequence similarity to epitopes (SDx data+IEDB database that contains epitopes that were previously experimentally verified as binding to MHC-I/II, derived from Listeria or other species), concomitantly, compute the probability of an epitope (e.g., using in-house ML model), and (iv) returns AA sequences of putative epitopes (FIG. 3) plus associated info (e.g., the protein they are derived from, location within the protein, associated info (e.g., source of the epitope), all of the metadata associated with the original DNA sequence).

The aptness of the sequence to become a vaccine is based on the desirability and suitability and its identification as being not cross reactive to any other species (host or pathogen).

### Example 2

### Antisense Oligo Development for Antimicrobial Resistance Genes

In this example, the goal is to identify sequences of import for the development of an anti-sense oligonucleotide (ASO)-based treatment intended to target all bacteria that carry a specific antibiotic resistance (AMR) gene variant, irrespective of their origin.

A subset of sequences (n≥1) is identified by Module 4A as unique to a gene conferring antibiotic resistance (AMR) from all sequences arriving in Module 4. Any bacteria can acquire a new AMR gene variant, either by acquiring the full gene through horizontal gene transfer (e.g., via plasmid) or changing their own pro-AMR gene repertoire (e.g., via point mutations/insertions/deletions, promoter changes, or gene duplications). Therefore, targeting a single AMR gene variant can impact many different bacterial species/strains/variants, as the emphasis is on function (e.g., the resistance to treatment with antimicrobials) that can be shared across pathogens because it confers an advantage. An ASO targeting that particular AMR gene variant will be useful in the treatment of all refractory infections that can be quickly assessed by a simple PCR.

Module B is then responsible for matching the sequence with the selection criteria, including desirability and suitability, used to identify candidates for ASO development.

Desirability is related to how useful it is to pursue ASO design for this AMR variant. As in Example 1, the desirability is dependent on the context in which the sequence is found, e.g., hard-to-treat, antibiotic-resistant strains, which is associated with the sequence itself as metadata. Here, the ML-driven selection of criteria applies especially to the estimate of desirability, as ML approaches have been very successful in predicting the possible functional consequences of AMR variants. The temporal and spatial information shows us the dynamic of how this sequence spreads, supporting the desirability to develop an ASO against it.

Suitability is related to what makes an effective ASO. For suitability Module B factors in the following: (i) the DNA sequence has to code for RNA, (ii) the RNA has to be important for the pathogen's function, (iii) the ASO should target the protein by affecting pre-mRNA maturation or mRNA translation, and/or (iv) the ASO should not have an off-target effect (e.g., should not bind to any other, unrelated sequences).

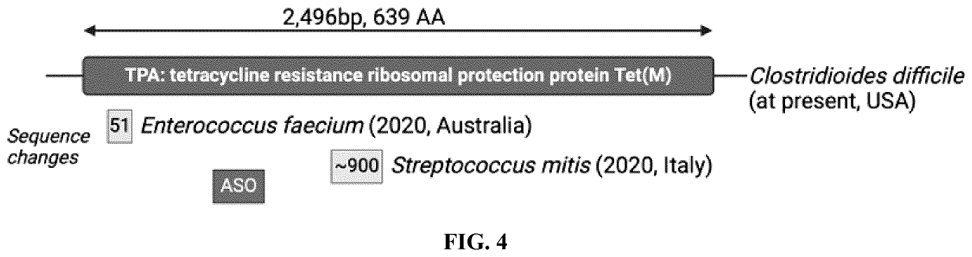

To illustrate the ASO design, a tetracycline resistance ribosomal protection protein Tet (M) (TPA) recently discovered in Clostridioides difficile in the USA was selected. Sequence analysis shows that similar sequences were found elsewhere, e.g., in a different strain of Clostridioides difficile (2022, Leiden and 2020, Australia), and different pathogens: Enterococcus faecium (2020, Australia), Streptococcus mitis (2020, Italy). To design an ASO against this AMR gene, the first third of the gene, where there are little-to-no sequence changes between the TPA sequences found in different bacterial species, was chosen. This ensures that the ASO will impact each of these different pathogens, restoring their sensitivity to tetracycline. In addition, it was confirmed that the 28-nt ASO is specific to this sequence (e.g., alignment score <40 for human), ensuring there are no undesirable off-target effects (e.g., safety issues). FIG. 4 shows the location of known DNA changes (that affect the AA sequence) and the approximate location of the ASO.

The aptness of the sequence to become a therapeutic ASO and restore tetracycline sensitivity is based on the desirability and suitability and its identification as being not cross reactive to any other species (host or pathogen).

### REFERENCES

Boolchandani, M.; D'Souza, A. W.; Dantas, G. Sequencing-Based Methods and Resources to Study Antimicrobial Resistance. Nat Rev Genet 2019. doi.org/10.1038/s41576-019-0108-4.
Immune Epitope Database & Tools. iedb.org.
Buthelezi, L. A.; Pillay, S.; Ntuli, N. N.; Gcanga, L.; Guler, R. Antisense Therapy for Infectious Diseases. Cells 2023, 12 (16), 2119. https://doi.org/10.3390/cells12162119.
Wan, Q.; Liu, X.; Zu, Y. Oligonucleotide Aptamers for Pathogen Detection and Infectious Disease Control. Theranostics 2021, 11 (18), 9133-9161. doi.org/10.7150/thno.61804.
Chakraborty, B.; Das, S.; Gupta, A.; Xiong, Y.; T-V, V.; Kizer, M. E.; Duan, J.; Chandrasekaran, A. R.; Wang, X. Aptamers for Viral Detection and Inhibition. ACS Infect. Dis. 2022, 8 (4), 667-692. doi.org/10.1021/acsinfecdis.1c00546.
Gupta, A.; Anand, A.; Jain, N.; Goswami, S.; Anantharaj, A.; Patil, S.; Singh, R.; Kumar, A.; Shrivastava, T.; Bhatnagar, S.; Medigeshi, G. R.; Sharma, T. K.; DBT India Consortium for COVID-19 Research. A Novel G-Quadruplex Aptamer-Based Spike Trimeric Antigen Test for the Detection of SARS-COV-2. Mol Ther Nucleic Acids 2021, 26, 321-332. doi.org/10.1016/j.omtn.2021.06.014.
Kim, T.-H.; Lee, S.-W. Aptamers for Anti-Viral Therapeutics and Diagnostics. IJMS 2021, 22 (8), 4168. doi.org/10.3390/ijms22084168.
Erickson, S.; Paulson, J.; Brown, M.; Hahn, W.; Gil, J.; Barron-Montenegro, R.; Moreno-Switt, A. I.; Eisenberg, M.; Nguyen, M. M. Isolation and Engineering of a Listeria Grayi Bacteriophage. Sci Rep 2021, 11 (1), 18947. doi.org/10.1038/s41598-021-98134-1.
NCBI. NCBI Datasets. www.ncbi.nlm.nih.gov/datasets/.
Johns Hopkins. Finding Datasets for Secondary Analysis. browse.welch.jhmi.edu/datasets/genomic-databases.
Balaji, A.; Kille, B.; Kappell, A. D.; Godbold, G. D.; Diep, M.; Elworth, R. A. L.; Qian, Z.; Albin, D.; Nasko, D. J.; Shah, N.; Pop, M.; Segarra, S.; Ternus, K. L.; Treangen, T. J. SeqScreen: Accurate and Sensitive Functional Screening of Pathogenic Sequences via Ensemble Learning. Genome Biol 2022, 23 (1), 133. doi.org/10.1186/s13059-022-02695-x.
Godbold, G. D.; Kappell, A. D.; LeSassier, D. S.; Treangen, T. J.; Ternus, K. L. Categorizing Sequences of Concern by Function To Better Assess Mechanisms of Microbial Pathogenesis. Infect Immun 2022, 90 (5), e0033421. doi.org/10.1128/IAI.00334-21.
Allen, J. E.; Gardner, S. N.; Slezak, T. R. DNA Signatures for Detecting Genetic Engineering in Bacteria. Genome Biol 2008, 9 (3), R56. doi.org/10.1186/gb-2008-9-3-r56.
Kulkarni, J. A.; Witzigmann, D.; Thomson, S. B.; Chen, S.; Leavitt, B. R.; Cullis, P. R.; Van Der Meel, R. The Current Landscape of Nucleic Acid Therapeutics. Nat. Nanotechnol. 2021, 16 (6), 630-643. doi.org/10.1038/s41565-021-00898-0.
Andres-Terre, M.; McGuire, H. M.; Pouliot, Y.; Bongen, E.; Sweeney, T. E.; Tato, C. M.; Khatri, P. Integrated, Multi-Cohort Analysis Identifies Conserved Transcriptional Signatures across Multiple Respiratory Viruses. Immunity 2015, 43 (6), 1199-1211. doi.org/10.1016/j.immuni.2015.11.003.
Mayhew, M. B.; Buturovic, L.; Luethy, R.; Midic, U.; Moore, A. R.; Roque, J. A.; Shaller, B. D.; Asuni, T.; Rawling, D.; Remmel, M.; Choi, K.; Wacker, J.; Khatri, P.; Rogers, A. J.; Sweeney, T. E. A Generalizable 29-mRNA Neural-Network Classifier for Acute Bacterial and Viral Infections. Nat Commun 2020, 11 (1), 1177. doi.org/10.1038/s41467-020-14975-w.
Fan, J.; Huang, S.; Chorlton, S. D. BugSeq: A Highly Accurate Cloud Platform for Long-Read Metagenomic Analyses. BMC Bioinformatics 2021, 22 (1), 160. doi.org/10.1186/s12859-021-04089-5.
GLOBAL BIODEFENSE STAFF. BugSeq Awarded BARDA DRIVe Funding to Develop Next-Gen Diagnostics for Any Respiratory RNA Virus. globalbiodefense.com/2022/05/03/bugseq-awarded-barda-drive-funding-to-develop-next-gen-diagnostics-for-any-respiratory-rna-virus/.

1-26. (canceled)

27. A computer implemented method comprising:
a) obtaining standardized and/or annotated genomic sequence fragments and sample metadata;
b) associating each genomic sequence fragment with one or more attributes; and
c) selecting sequences in which the one or more attributes and/or sample metadata fulfill intent-specific criteria.

28. The method of claim 27, wherein the genomic sequence fragments are derived from a non-human animal sample.

29. The method of claim 27, wherein the genomic sequence fragments are from one or more microorganisms and/or viruses and/or a host from which the sample is derived.

30. The method of claim 29, wherein the one or more microorganisms and/or viruses comprises an emerging microorganism and/or virus.

31. The method of claim 27, wherein the sample metadata comprises demographic information, health information, environmental information, or any combination thereof.

32. The method of claim 27, wherein the attributes are associated with each genomic sequence fragment based on data from known databases, inferred or predicted attributes, or contemporary analysis.

33. The method of claim 27, wherein the attributes comprise one or more of: level of uniqueness as compared to other sequences or genomes, source organism, list of organisms or species which contain sequence, environment in which sequence was obtained, identification of sequence motifs contained within the genomic sequence fragment, and fitness for prophylactic or therapeutic use.

34. The method of claim 27, wherein the intent is a therapeutic or prophylactic treatment.

35. The method of claim 27, wherein the intent comprises development and/or identification of vaccines, antisense oligonucleotides (ASOs), aptamers, reporter genes, natural antagonists to combat pathogens, cis-acting elements, cis-regulatory elements, operons, tertiary structures, organelle targeting sequences, mRNA circularization elements, synthetic barcodes, drug tolerance/resistance genes, GMO signatures, transposon landing sites, regulatory non-coding RNAs, or a combination thereof.

36. The method of claim 27, wherein selecting sequences comprises analyzing the uniqueness of the sequence, desirability for the intent, and suitability of the intent based on the one or more attributes and sample metadata.

37. The method of claim 27, wherein any one or more or all of steps a), b), or c) utilizes an artificial intelligence and machine learning (AI/ML) system.

38. The method of claim 27, further comprising the step of synthesizing a therapeutic molecule based on a selected sequence.

39. The method of claim 38, further comprising the step of administering the therapeutic molecule to a subject.

40. A computer-implemented method for classifying biological data, comprising one or more of the steps of: a) receiving an offline dataset partitioned into training and test sets; b) transforming dataset features into sparse high-dimensional vectors using a bag-of-words encoding of biological or clinical elements; c) generating multiple random balanced subsets of the training data by under-sampling a majority class to equalize class representation; d) training a plurality of classifiers on respective balanced subsets; e) aggregating outputs of the classifiers to obtain a consensus classification; and f) applying the consensus classification to biological samples in the test dataset.

41. A system comprising a processor running software configured to carry out the method of claim 40.

42. The system of claim 41, wherein the system is configured to carry out each of the steps of the method.

43. The system of claim 41, wherein the system is configured to carry out each of the steps concomitantly and in real-time.

44. The system of claim 41, further comprising a sample processing component.

45. The system of claim 44, further comprising a sample analysis component.

46. The system of claim 45, wherein the sample analysis component comprises an automated nucleic acid sequencing component.
